(19) United States
(12) Patent Application Publication
(10) Pub. No.: US 2008/0183292 A1
(43) Pub. Date: Jul. 31, 2008

(54) COMPLIANT INTERVERTEBRAL PROSTHETIC DEVICES EMPLOYING COMPOSITE ELASTIC AND TEXTILE STRUCTURES

(75) Inventor: Hai H. TRIEU, Cordova, TN (US)

Correspondence Address: HESLIN ROTHENBERG FARLEY & MESITI P.C., 5 COLUMBIA CIRCLE, ALBANY, NY, 12203, US

(73) Assignee: WARSAW ORTHOPEDIC, INC., Warsaw, IN (US)

(21) Appl. No.: 11/668,116

(22) Filed: Jan. 29, 2007

(51) Int. Cl. A61F 2/44

(52) U.S. Cl. 623/17.11

(57) ABSTRACT

An intervertebral prosthetic device is provided for implanting within an intervertebral disc space between first and second vertebral bodies. The device includes a body component and a core component, one of which is an elastic-material structure and the other of which is a composite structure, including a textile structure embedded within an elastic material. The composite structure has a higher compressive modulus of elasticity than the elastic-material structure to enhance device support when in an operable position within the intervertebral disc space. In various embodiments, a porous textile structure partially covers the body component to, for example, facilitate bony in-growth or soft tissue in-growth into the device and therefore fixation of the device when in operable position within the intervertebral disc space.

### CROSS-REFERENCE TO RELATED APPLICATIONS

This application contains subject matter which is related to the subject matter of the following applications, which are hereby incorporated herein by reference in their entirety:

“In Situ Formation of Intervertebral Disc Implants”, Hai H. Trieu, U.S. Ser. No. 10/970,462, filed Oct. 21, 2004, and published on Apr. 27, 2006 as U.S. Patent Application No. U.S. 2006/0089719 A1;

“Hybrid Intervertebral Disc System”, Hai H. Trieu, U.S. Ser. No. 10/765,260, filed Jan. 27, 2004, and published on Jul. 28, 2005 as U.S. Patent Application Publication No. US 2005/0165485 A1;

“Instruments And Methods For Implanting Nucleus Replacement Material In An Intervertebral Disc Nucleus Space”, U.S. Ser. No. 11/343,088, filed Jan. 30, 2006;

“Intervertebral Prosthetic Disc”, Heinz et al., U.S. Ser. No. 11/343,935, filed Jan. 31, 2006; and

“Compliant Intervertebral Prosthetic Devices with Motion Constraining Tethers”, Hai H. Trieu, U.S. Ser. No. 11/616,388, filed Dec. 27, 2006.

### TECHNICAL FIELD

The present invention relates generally to spinal implants and methods, and more particularly, to intervertebral prosthetic joint devices and methods for use in total or partial replacement of a natural intervertebral disc.

### BACKGROUND OF THE INVENTION

In the treatment of disease, injuries and malformations affecting spinal motion segments, and especially those affecting disc tissue, it has been known to remove some or all of a degenerated, ruptured or otherwise failing disc. In cases involving intervertebral disc tissue that has been removed, or is otherwise absent from a spinal motion segment, corrective measures are typically desirable.

In one approach, adjacent vertebrae are fused together using transplanted bone tissue, an artificial fusion component, or other compositions or devices. Spinal fusion procedures, however, have raised concerns in the medical community that the biomechanical rigidity of the intervertebral fusion may predispose neighboring spinal motion segments to rapid deterioration. Unlike a natural intervertebral disc, spinal fusion prevents the fused vertebrae from pivoting and rotating with respect to one another. Such lack of mobility tends to increase stress on adjacent spinal motion segments. Additionally, conditions may develop within adjacent spinal motion segments, including disc degeneration, disc herniation, instability, spinal stenosis, spondylosis and facet joint arthritis as a result of the spinal fusion. Consequently, many patients may require additional disc removal and/or another type of surgical procedure as a result of the spinal fusion. Alternatives to spinal fusion are therefore desirable.

Alternative approaches to bone grafting employ a manufactured implant made of a synthetic material that is biologically compatible with a body in the vertebrae. There have been extensive attempts at developing acceptable prosthetic implants that can be used to replace an intervertebral disc and yet maintain the stability and range of motion of the intervertebral disc space between adjacent vertebrae. While many types of prosthetic devices have been proposed, there remains a need in the art for further enhanced intervertebral prosthetic disc devices.

### SUMMARY OF THE INVENTION

The shortcomings of the prior art are overcome and additional advantages are provided, in one aspect, through provision of an intervertebral prosthetic device which includes a body component configured for implantation within an intervertebral space defined between a first vertebral body and a second vertebral body. The body component is a composite structure including a textile structure embedded within an elastic material. The prosthetic device further includes a core component disposed within the body component. The core component includes one of a spherical-shaped elastic structure or a cylindrical-shaped elastic structure, and wherein the body component has a higher compressive modulus of elasticity than the core component to enhance device support when the intervertebral prosthetic device is in operable position within an intervertebral space between the first and second vertebral bodies.

In another aspect, an intervertebral prosthetic device is provided which includes a body component and a core component. The body component is configured for implantation within an intervertebral space defined between a first vertebral body and a second vertebral body, and the core component is disposed within the body component. The core component includes a cylindrical-shaped structure having a longitudinal axis extending in a direction which intersects endplates of the first vertebral body and the second vertebral body when the intervertebral prosthetic device is disposed in operable position within the intervertebral space. One of the core component and the body component is an elastic structure and the other of the core component and body component is a composite structure. The composite structure includes a textile structure embedded within an elastic material, and has a higher compressive modulus of elasticity than the elastic structure to enhance device support when in operable position within the intervertebral space between a first vertebral body and a second vertebral body.

In a further aspect, an intervertebral prosthetic device is provided which includes a body component comprising an elastic structure having a first side, a first end, a second side, a second end, an upper surface and a lower surface, wherein the upper surface and the lower surface are disposed in opposing relation to a respective endplate of a first vertebral body and a second vertebral body defining an intervertebral space when the intervertebral prosthetic device is in operable position within the intervertebral space. A composite structure wraps around the body component to cover the first side, first end, second side and second end thereof, with the upper surface and lower surface of the body being component uncovered by the composite structure, wherein the composite structure has a higher compressive modulus of elasticity than the elastic structure to enhance device support when the intervertebral prosthetic device is in operable position within the intervertebral space between the first and second vertebral bodies with the upper and lower surfaces of the body component in opposing relation to the endplates of the first and second vertebral bodies.

In another aspect, an intervertebral prosthetic device is provided which includes a body component having at least a first end and a second end, and comprising an elastic structure. Multiple composite structures are provided, with one composite structure being disposed at the first end and another composite structure being disposed at the second end of the body component. Each composite structure includes a textile structure embedded within an elastic material. The multiple composite structures have a higher compressive modulus of elasticity than the body component to provide enhanced device support when the intervertebral prosthetic device is in an operable position within an intervertebral space between a first vertebral body and a second vertebral body.

In yet another aspect, an intervertebral prosthetic device is provided which includes an elastic core component and a composite structure at least partially surrounding the elastic core component. The composite structure, which includes a textile structure embedded within an elastic material, has a higher compressive modulus of elasticity than the elastic core component to enhance device support when in operable position within an intervertebral space between a first vertebral body and a second vertebral body. The prosthetic device further includes a porous textile structure at least partially covering the composite structure, and having a different compressive modulus of elasticity than the composite structure. The porous textile structure, composite structure and elastic core component are configured to facilitate implantation of the intervertebral prosthetic device within the intervertebral space between the first and second vertebral bodies in an operable position with at least a portion of the porous textile structure contacting at least one endplate of the first vertebral body and second vertebral body defining the intervertebral space.

In still another aspect, an intervertebral prosthetic device is provided which includes a porous textile structure having at least a first end and a second end, and multiple composite structures. One composite structure of the multiple composite structures is disposed at the first end of the porous textile structure and another composite structure of the multiple composite structures is disposed at the second end of the porous textile structure. Each composite structure, which includes a porous textile structure embedded within an elastic material, has a different compressive modulus of elasticity than the porous textile structure to enhance device support when in an operable position within an intervertebral space between a first vertebral body and a second vertebral body.

In a further aspect, an intervertebral prosthetic device is provided which includes an elastic body component configured for implantation within an intervertebral space defined between a first vertebral body and a second vertebral body. The prosthetic device further includes at least one composite structure extending through the elastic body component between a first surface and a second surface thereof. Each composite structure includes a textile structure embedded within an elastic material. The at least one composite structure has a higher compressive modulus of elasticity than the elastic body component to enhance device support when in an operable position within the intervertebral space between the first and second vertebral bodies.

In a yet further aspect, an intervertebral prosthetic device is provided which includes a load-bearing elastic body component having shape memory. The elastic body component is in a first, folded configuration effective to serve as a prosthetic disc nucleus, and is configurable into a second, straightened configuration for insertion through an opening in an intervertebral disc annulus fibrosis. The shape memory is effective to return the elastic body component to its first, folded configuration after the elastic body component is straightened to its second, straightened configuration and inserted into an intervertebral space. Multiple composite structures are provided, with one composite structure being disposed at a first end of the elastic body component and another composite structure being disposed at a second end of the elastic body component. Each composite structure includes a textile structure embedded within an elastic material. The composite structures enhance device support when in an operable position within an intervertebral space between a first vertebral body and a second vertebral body.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 depicts a lateral view of a portion of a human vertebral column;

FIG. 2 depicts a lateral view of a pair of adjacent vertebrae of a vertebral column;

FIG. 3 is a top sectional plan view of a vertebra;

FIG. 4 is a lateral view of a portion of a vertebral column posteriorally receiving an intervertebral implant, in accordance with an aspect of the present invention;

FIG. 5 is a lateral sectional view of an intervertebral disc space and intervertebral prosthetic device implanted therein, in accordance with an aspect of the present invention;

FIGS. 6A-6C are top sectional views of an intervertebral disc space and alternate embodiments of an intervertebral prosthetic device, in accordance with an aspect of the present invention;

FIG. 7 is a top sectional view of an intervertebral disc space with bilateral, posteriorally-inserted intervertebral prosthetic devices implanted therein, in accordance with an aspect of the present invention;

FIG. 8 is a top sectional view of an intervertebral disc space illustrating an alternate embodiment of bilateral, posteriorally-inserted intervertebral prosthetic devices implanted therein, in accordance with an aspect of the present invention;

FIGS. 9A & 9B are top sectional views of an intervertebral disc space with alternate embodiments of an intervertebral prosthetic device implanted therein, in accordance with an aspect of the present invention;

FIG. 10 is a top sectional view of an intervertebral disc space with a further embodiment of bilateral, posteriorally-inserted intervertebral prosthetic devices implanted therein, in accordance with an aspect of the present invention;

FIG. 11 is a top sectional view of an intervertebral disc space showing an alternate embodiment of an intervertebral prosthetic device, in accordance with an aspect of the present invention;

FIG. 12 is a top sectional view of an intervertebral disc space showing another embodiment of an intervertebral prosthetic device, in accordance with an aspect of the present invention;

FIG. 13 is a lateral sectional view of an intervertebral disc space and one embodiment of an intervertebral prosthetic device implanted therein, in accordance with an aspect of the present invention;

FIG. 14 is a lateral sectional view of an intervertebral disc space and another embodiment of an intervertebral prosthetic device implanted therein, in accordance with an aspect of the present invention;

FIG. 15 is a lateral sectional view of an intervertebral disc space and another embodiment of an intervertebral prosthetic device implanted therein, in accordance with an aspect of the present invention; and

FIG. 16 is a lateral sectional view of an intervertebral disc space and another embodiment of an intervertebral prosthetic device implanted therein, in accordance with an aspect of the present invention.

### BEST MODE FOR CARRYING OUT THE INVENTION

The present invention relates generally to vertebral reconstructive devices, and more particularly, to a functional intervertebral prosthetic disc device and related methods of implantation. For purposes of promoting an understanding of the principles of the invention, reference is made below to the embodiments, or examples, illustrated in the drawings and specific language is used to describe the same. It will nevertheless be understood that no limitation on the scope of the invention is thereby intended. Any alternations and further modifications in the described embodiments, and any further applications of the principles of the invention as described herein are contemplated as would normally occur to one skilled in the art to which the invention relates.

In human anatomy, the spine is a generally flexible column that can take tensile and compressive loads. The spine also allows bending motion and provides a place of attachment for tendons, muscles and ligaments. Generally, the spine is divided into three sections: the cervical spine, the thoracic spine and the lumbar spine. The sections of the spine are made up of individual bones called vertebrae. The vertebrae are separated by intervertebral discs, which are situated between adjacent vertebrae.

The intervertebral discs function as shock absorbers and as joints. Further, the intervertebral discs absorb the compressive and tensile loads to which the spinal column may be subjected. At the same time, the intervertebral discs allow adjacent vertebral bodies to move relative to each other a limited amount, particularly during bending, or flexure, of the spine. Thus, the intervertebral discs are under constant muscular and/or gravitational pressure and generally are the first parts of the lumbar spine to show signs of deterioration.

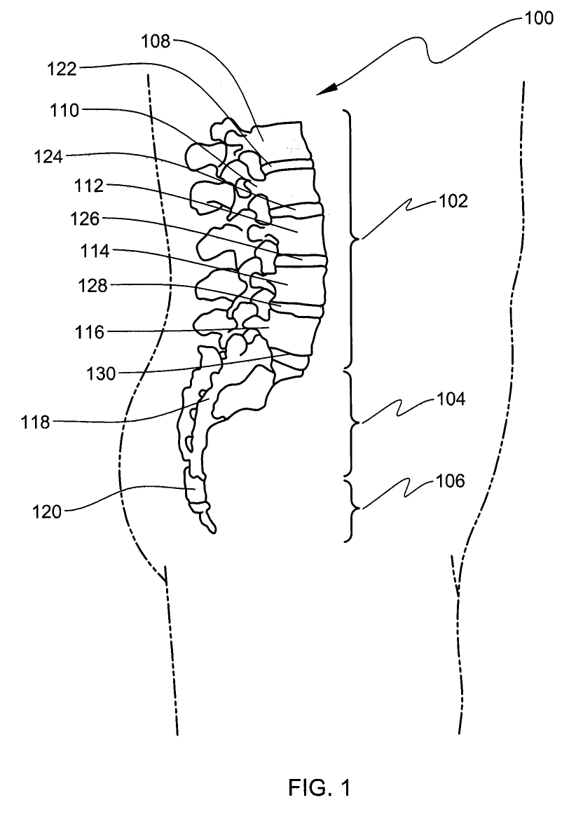

Referring now to the figures, and initially to FIG. 1, a portion of a vertebral column 100 is shown. As depicted, vertebral column 100 includes a lumbar region 102, a sacral region 104, and a coccygeal region 106. As is known in the art, vertebral column 100 also includes a cervical region and a thoracic region. For clarity and ease of discussion, the cervical region and the thoracic region are not illustrated.

As shown in FIG. 1, lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As also depicted in FIG. 1, a first intervertebral lumbar disc 122 is disposed between first lumbar vertebra 108 and second lumbar vertebra 110. A second intervertebral lumbar disc 124 is disposed between second lumbar vertebra 110 and third lumbar vertebra 112. A third intervertebral lumbar disc 126 is disposed between third lumbar vertebra 112 and fourth lumbar vertebra 114. Further, a fourth intervertebral lumbar disc 128 is disposed between fourth lumbar vertebra 114 and fifth lumbar vertebra 116. Additionally, a fifth intervertebral lumbar disc 130 is disposed between fifth lumbar vertebra 116 and sacrum 118.

In one particular embodiment, if one of the intervertebral lumbar discs 122, 124, 126, 128, 130 is diseased, degenerated, damaged, or otherwise in need of replacement, that intervertebral lumbar disc can be at least partially removed and replaced with an intervertebral prosthetic disc according to one or more of the embodiments described herein. In one embodiment, a portion of the intervertebral lumbar disc 122, 124, 126, 128, 130 is removed via a discectomy, or similar surgical procedure, well known in the art. Further, removal of intervertebral lumbar disc material can result in the formation of an intervertebral disc space (not shown) between two adjacent lumbar vertebrae.

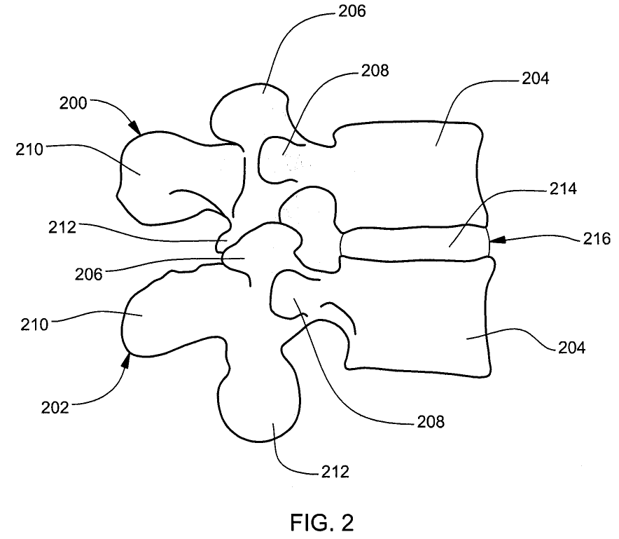

FIG. 2 depicts a detailed lateral view of two adjacent vertebra, e.g., two of the lumbar vertebra 108, 110, 112, 113, 116 shown in FIG. 1. In particular, FIG. 2 illustrates a superior vertebra 200 and an inferior vertebra 202. Each vertebra 200, 202 includes a vertebral body 204, a superior articular process 206, a transverse process 208, a spinous process 210 and an inferior articular process 212. FIG. 2 further illustrates that an intervertebral disc space 214 can be established between superior vertebra 200 and inferior vertebra 202 by removing the intervertebral disc 216. As described in greater detail below, an intervertebral prosthetic device, according to one or more of the embodiments described herein, can be inserted into intervertebral disc space 214 between superior vertebra 200 and inferior vertebra 202.

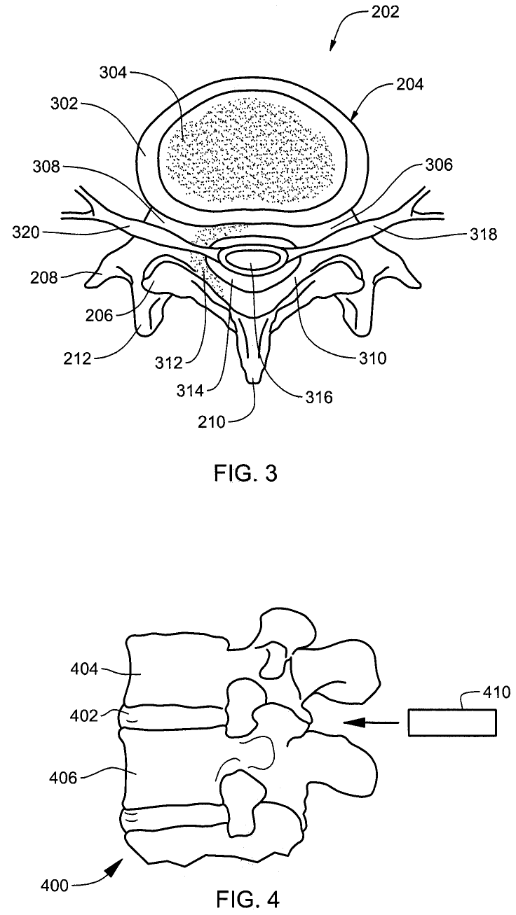

Referring to FIG. 3, a vertebra, e.g., inferior vertebra 202 (of FIG. 2), is illustrated in top plan view. As shown, vertebral body 204 of inferior vertebra 202 includes a cortical rim 302 composed of cortical bone. Also, vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, i.e., those structures described above in conjunction with FIGS. 2 & 3. The first and second cervical vertebrae are structurally different than the rest of the vertebrae in order to support the skull.

FIG. 4 illustrates a vertebral joint 400 which includes an intervertebral disc 402 extending between vertebrae 404, 406. Disc 402 may be partially or entirely removed and an intervertebral implant 410 inserted between the vertebrae 404, 406 to preserve motion within joint 400. Although the illustration of FIG. 4 generally depicts vertebral joint 400 as a lumbar vertebral joint, it should be understood that the devices and methods of this disclosure are applicable to all regions of a vertebral column, including the cervical and thoracic regions. Additionally, although the illustration of FIG. 4 generally depicts a posterior approach for insertion of implant 410, other approaches, such as a lateral or anterior approach, may alternatively be employed.

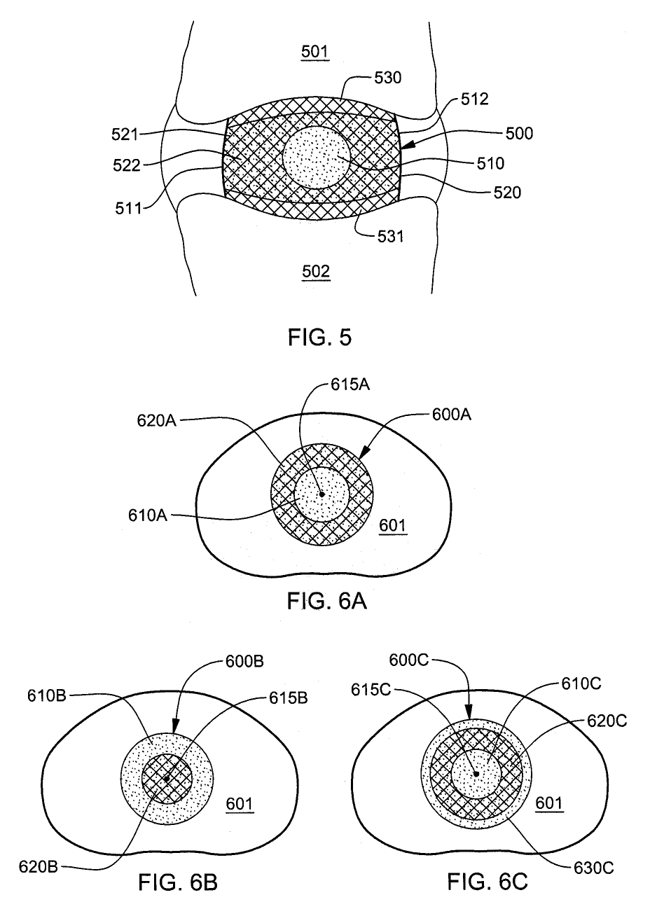

FIGS. 5-16 detail various intervertebral prosthetic device configurations, in accordance with aspects of the present invention. In each embodiment, a composite structure comprising a textile structure embedded within an elastic material is employed to strengthen the intervertebral prosthetic device. Specifically, the composite structure has a higher compressive modulus of elasticity than an elastic component or region (such as a body or core component) of the prosthetic device to enhance device support when in an operable position within an intervertebral disc space between adjacent first and second vertebral bodies.

Referring first to the embodiment of FIG. 5, an intervertebral prosthetic device 500 is shown implanted within an intervertebral disc space between a superior vertebral body 501 and an inferior vertebral body 502. Intervertebral prosthetic device 500 includes an elastic core component 510, which in this embodiment is a spherical-shaped elastic structure (e.g., having a diameter in the range of 4-14 mm). This spherical-shaped elastic structure is surrounded by a composite structure 520 (e.g., body component) comprising a textile structure 521 embedded within an elastic material 522. The composite structure has a higher compressive modulus of elasticity than the elastic core component, and thus provides enhanced support to the intervertebral prosthetic device, for example, in the regions of a first end 511 and a second end 512 thereof. As a specific example, considering the elastic material of the composite structure and the elastic core component together, the textile structure of the composite structure is embedded in at least 25% of the elastic material.

A first porous textile structure 530 and a second porous textile structure 531 are disposed above and below the composite structure 520 to interface with and facilitate coupling of the intervertebral prosthetic device to the superior vertebral body 501 and inferior vertebral body 502, respectively, as explained further below. As explained below, first and second porous textile structures 530, 531 may have a thickness in a range of 1-5 mm (for example) and comprise the same textile structure as textile structure 521 of composite structure 520, or a different textile structure, either in terms of material or fabrication. By way of example, first and second porous textile structures 530, 531 may be constructed with a higher compressive modulus of elasticity (i.e., greater rigidity) than composite structure 520.

In one embodiment, intervertebral prosthetic device 500 is rectangular-shaped, however, as described further below, other shapes such as kidney, oval, oblong, semi-circular, semi-toroidal, trapezoidal, triangular, spherical, ellipsoidal, capsule, etc., are also contemplated (either with or without convex upper and lower surfaces).

The implant process includes (in one embodiment) creating an incision in a patient's back and forming a posterior, unilateral opening on one or both lateral sides of the intervertebral disc space. Each opening may be any size required to accept a single intervertebral prosthetic device configured as described herein. For example, an 11 mm opening may be suitable. Through this opening, instrumentation may be inserted to evacuate remaining disc tissue. Instrumentation may also be inserted to mill or otherwise dislocate bone to fashion a path, track or recess in one or both of the vertebral endplates adjacent to the intervertebral disc space. It is understood that in certain embodiments, no bone removal may be needed. The disc space may be extracted through the milling procedure and/or subsequent insertion procedures.

Elastic core component 510 is a unitary elastic structure (in one embodiment) formed from one or more resilient materials which have a lower compressive modulus of elasticity than the composite structure. Suitable flexible core materials may include polymeric elastomers such as polyolefin rubbers; polyurethanes (including polyetherurethane, polycarbonate urethane, and polyurethane with or without surface modified endgroups); copolymers of silicone and polyurethane with or without surface modified endgroups; silicones; and hydrogels. Polyisobutylene rubber, polyisoprene rubber, neoprene rubber, nitrile rubber, and/or vulcanized rubber of 5-methyl-1,4-hexadiene may also be suitable.

Composite structure 520 may be formed of any suitable combination of textile structure and elastic material wherein the textile structure can be embedded within the elastic material. As used herein, the phrase “textile structure” includes any woven, non-woven, knitted, braided, etc. structure wherein the filaments or fibers may be fabric, polymeric, ceramic, metallic, etc. More particularly, the textile structures can be made of yarns or fibers of any biocompatible (one or more) materials, including polyester such as polyethyleneterephthalate (PET), polyethylene such as ultra-high molecular-weight polythylene (UHMWPE), polyaryletherketone such as polyetheretherketone (PEEK), polypropylene, polyamide, acetate, acrylic, aramid, elastoester, polybenzimidazole, etc. Elastic material 522 may be the same one or more elastic materials as employed in elastic core component 510, or one or more different elastic materials, for example, chosen from the above-noted list of resilient materials. In certain embodiments, it may be advantageous to employ an elastic material 522 which has a higher compressive modulus of elasticity than that of elastic core component 510. In certain other embodiments, it may be advantageous to employ an elastic material 522 within composite structure 520 which has a lower compressive modulus of elasticity than that of elastic core component 510.

Depending on the required mechanical properties, load support and/or allowable motions, the textile structure within the composite structure can be produced using one or more methods such as weaving, knitting, braiding, heat setting, heat bonding, laminating, etc. The textile structure is a three-dimensional structure with voids or porosity varying from 10 to 3,000 microns. As a specific example, porosity might vary between 100 and 1,000 microns. As a further enhancement, the textile structure may be surface-modified, for example, using plasma treatment, to facilitate and improve adhesion of the elastomeric material(s) within which the textile structure is embedded.

Embedding of textile structure 521 within elastic material 522 can be achieved in a number of processes. For example, the composite structure can be produced by obtaining a porous textile structure (characterized as noted above) and then injecting an elastic material, such as an elastomer, into the textile structure to flush out air and fill the porous structure (and any center void therein). As noted, the elastic material employed within the composite structure may be the same material as the elastic region, or different. After flushing out the textile structure, the elastic material is allowed to solidify, thereby producing the composite structure. An advantage of the composite structure is that a higher compressive modulus of elasticity material can be formed integral with the elastic core component, thereby providing a higher modulus elastic body component surrounding the elastic core component. Suitable textile structures include any biocompatible material capable of being embedded within an elastic material, such as those noted above.

As an alternative manufacturing approach, a three-dimensional (3-D) porous textile structure could be placed in a mold, within which a flowable/self-curable pre-cursor material is injected to form a void-free composite structure. The precursor material is allowed to cure and become an elastomer to form the final structure. As a variations on this approach, the precursor material could be heat-curable or light-curable. As a further enhancement, the resultant composite structure could be inserted into an outer porous textile jacket to form the final prosthetic device. Still further, the resulting composite structure could further be embedded in an outer layer of elastomeric material to form the final prosthetic device. As another implementation, a spherical elastomer material could be molded, and then inserted into the center of a textile structure, which is then molded via injection of a flowable/self-curable precursor material to form the void-free composite structure after curing thereof. Further embodiments described hereinbelow can be readily created using various ones of the steps described in the above examples.

Porous textile structures 530, 531 may be formed from any biocompatible textile structure, and may comprise the same textile structure as textile structure 521 employed within composite structure 520, or a different textile structure. If the same textile structure is employed, then layers 530, 531 can be achieved in the above-described fabrication process by utilizing a soluble material within the upper and lower surfaces of the textile structure. For example, a soluble material could extend 1-5 mm into the structure from the upper and lower surfaces thereof, which after solidification of the elastic material injected into the textile structure, may be removed by soaking the upper and lower surfaces in a warm water solution to dissolve out the soluble material. After drying of the resultant structure, the intervertebral prosthetic device illustrated in FIG. 5 is obtained.

As noted, porous textile structures 530, 531 may be a different textile structure than textile structure 521 employed within composite structure 520. For example, it may be advantageous to have porous textile structures 530, 531 be more rigid than composite structure 520 by employing a different structural pattern and/or different materials. If the two textile structures are different construction or materials, then they may be secured together using various techniques, such as adhesive bonding or laminating, or braiding or weaving techniques to stitch the textile structures together. Porosity of the textile structures 530, 531 advantageously allows for bony in-growth from the endplates of the first and second vertebral bodies when the intervertebral prosthetic device is disposed within an intervertebral disc space. Bony in-growth into porous textile structures 530, 531 may be enhanced by coating the structures with a biocompatible and osteoconductive material such as hydroxyapatite (HA), tricalcium phosphate (TCP), and/or calcium carbonate to promote bone in-growth and fixation. Alternatively, osteoinductive coatings, such as proteins from transforming growth factor (TGF), beta superfamily, or bone-morphogenic proteins, such as BMP2 or BMP7, may be used.

In operation, the intervertebral prosthetic device elastically deforms under compressive loads and elastically stretches in response to a force which may pull the fabric layers away from one another. The intervertebral prosthetic device may also deform or flex under flexion-extension or lateral bending motion. The composite structure advantageously reinforces the intervertebral prosthetic device for enhanced operation of the prosthesis responsive to one or more of these motions.

Numerous reconfigurations of the prosthetic device of FIG. 5 are possible, as explained further below in connection with the illustrated embodiments of FIGS. 6A-16. Further enhancements may include varying the textile structure pattern, density or material(s) either within composite structure 520, or between porous textile structures 530, 531 and composite structure 520. For example, porosity or spacing between the fibers or filaments forming the different structures may be varied by varying the weave pattern. As one example, porosity of textile structure 521 of composite structure 520 can vary from first end 511 to second end 512 thereof. By varying porosity, the amount of elastic material embedded within the textile structure can vary, thereby producing a structure of varying modules. By way of example, if an anterior portion of the intervertebral prosthetic device requires less support, then a less dense textile structure can be employed within the composite structure in the anterior region of the device compared with the posterior region thereof.

Further, if different or multiple materials are used for the porous textile structure and/or the textile structure of the composite structure, then one of the materials could be selected as or integrated with an x-ray marker, such as a tantalum marker, to assist in positioning of the intervertebral prosthetic device when disposed within a disc space. For example, several wire filaments could be threaded around the periphery of the device, either within the porous textile structure or the composite structure, to enable a surgeon to view the general outline of the device in situ.

As a further variation, elastic material 522 within which textile structure 521 is embedded can have a varying composition or varying porosity from, for example, a first end to a second end of the prosthetic device. Progressively changing compressive modulus of elasticity from one end to the other end can be achieved by a number of techniques. For example, controlled reactive injection molding could be employed to inject different levels of cross-linking material into the elastic material during formation of the composite structure. That is, a progressively higher amount of cross-linking could be employed from the first end to the second end of the composite structure, resulting in a progressively changing modulus from a lower modulus end to a higher modulus end. As one example, less cross-linking may be employed at an anterior end of the intervertebral prosthetic device, and more cross-linking at a posterior end thereof.

As a further approach, two or more different elastic materials may be mixed when forming the composite structure (or the elastic core), with one material having a higher compressive modulus of elasticity than the other material(s). In this approach, the concentrations of the materials can be progressively varied as the materials are injected into the textile structure 521, with (for example) the higher modulus material having a higher concentration near the posterior end of the intervertebral prosthetic device, and the lower modulus material having a higher concentration near the anterior end thereof.

Variations in porosity can also be employed to achieve regions of different elasticity within the composite structure, or within the elastic core. For example, porosity of the composite structure may decrease from a first end to a second end thereof to achieve an at least partially progressively increasing modulus from the first end to the second end of the prosthetic device. In certain other embodiments (e.g., where the elastic region forms the body component of the prosthetic device), porosity of the elastic region may vary, for example, from the ends of the device towards the middle of the device, or from a first end to a second end thereof.

Further, one or more regions, in addition to elastic core 510 and composite structure 520, could be formed, for example, either within the composite structure or within the elastic core. These one or more additional regions could have a common geometric shape, and reduce in size from the first end to the second end of the prosthetic device. Further, such one or more regions could have a material with a different compressive modulus of elasticity than the balance of the composite structure or elastic core and may be used to control, adjust or modify the hardness, stiffness, flexibility, or compliance of the composite structure. These one or more regions may be discrete bodies within the composite structure or have a gradient quality which allows the regions to blend into the composite structure between the first end and the second end. By way of example, the one or more regions could comprise defined voids within the composite structure or within the elastic core.

These one or more additional regions may be formed of materials different from the elastic core component or from the elastic material of the composite structure. These materials may be stiffer or more pliable than the material used in the elastic core or the composite structure. Further, if the regions are voids, then in certain embodiments, one or more of these voids may function as reservoirs for therapeutic agents such as analgesics, anti-inflammatory substances, growth factors, antibiotics, steroids, pain medications, or combinations of agents. Growth factors may comprise any members of the families of transforming growth factor beta (TGH-beta), bone morphogenic proteins (BMPs), recombinant human bone morphogenic proteins (rh BMPs), insulin-like growth factors, platelet-derived growth factors, fibroblast growth factors, or any other growth factors that help promote tissue repair of surrounding tissues.

Additionally, one or more of the above-noted therapeutic agents could be included within the porous textile structures 530, 531, along with or in place of osteoconductive material or osteoinductive coatings.

Implantation of the prosthetic device can be performed anteriorally, laterally, or posteriorally. A posterior approach may offer a surgeon a technique similar to fusion with which the surgeon may already be familiar. The posterior approach may allow herniations impinging on a nerve root to be more easily decompressed. Further, later revision surgeries may be more easily managed compared to anteriorally placed devices. However, a lateral or anterior approach could be employed depending upon the prosthetic device to be implanted. Depending upon the implantation approach, various characteristics of the prosthetic device can be chosen, such as configuration and size. As a further enhancement, initial fixation may be achieved using screws, pins, etc. (not shown) to anchor the intervertebral prosthetic device in fixed position prior to bony in-growth into the prosthetic device.

Note that relative dimensions of the prosthetic device embodiments of FIGS. 5-16 are exemplary only. Further, in practice, the intervertebral prosthetic devices may be sized larger or smaller relative to the intervertebral disc space, as desired. For example, in certain embodiments, it may be desirable to substantially completely fill the intervertebral disc space with the prosthetic device implant.

As noted, FIGS. 6A-16 depict various alternate embodiments of an intervertebral prosthetic device, in accordance with aspects of the present invention. Although depicted as different configurations, unless otherwise noted, the elastic region, composite structure and porous textile structures are assumed herein to be identical to those described above in connection with the intervertebral prosthetic device of FIG. 5, including any of the noted variations thereof. For purposes of clarity, these materials, compositions and variations are not expressly repeated below for each embodiment. For these aspects, reference should be made to the above-noted discussion of the prosthetic device of FIG. 5 and its variations.

FIG. 6A depicts an alternate embodiment of an intervertebral prosthetic device 600A, in accordance with an aspect of the present invention. Intervertebral prosthetic device 600A, shown disposed within an intervertebral disc space 601 defined between adjacent vertebral bodies (not shown), includes a core component 610A comprising an elastic only region, which is partially surrounded by a composite structure 620A. Core component 610A comprises an elastic material similar to that described above in connection with elastic core component 510 of the embodiment of FIG. 5, while composite structure 620A is a composite structure, such as composite structure 520 described above in connection with the embodiment of FIG. 5. Specifically, composite structure 620A is a textile structure embedded within an elastic material, as described above. In this embodiment, core component 610A is a solid, cylindrical-shaped structure extending along a longitudinal axis 615A, and composite structure 620A is a ring-shaped structure surrounding core component 610A along its longitudinal axis 615A. As shown in FIG. 6A, the upper surface (and the lower surface) of the cylindrical-shaped core 610A and composite structure 620A are exposed and in opposing relation to a respective endplate of the first and second vertebral bodies defining the intervertebral disc space 601.

FIG. 6B illustrates an alternative embodiment of an intervertebral prosthetic device 600B which is similar to intervertebral prosthetic device 600A of FIG. 6A, with the exception that the elastic region and the composite structure are reversed. That is, in intervertebral prosthetic device 600B of FIG. 6B, a composite structure 620B is a cylindrical-shaped core component, while elastic region 610B is a ring-shaped structure which surrounds composite structure 620B along its longitudinal axis 615B. In this embodiment, the upper and lower surfaces of composite structure 620B and elastic region 610B are exposed, and in opposing relation to the endplates of the first and second vertebral bodies defining intervertebral disc space 601.

FIG. 6C depicts a further variation on the intervertebral prosthetic device of FIG. 6A. In this embodiment, the intervertebral prosthetic device 600C again includes a cylindrical-shaped core 610C comprising an elastic only region extending along a longitudinal axis, and ring-shaped composite structure 620C comprising a body region surrounding the core region. Additionally, the prosthetic device includes an elastic shell 630C surrounding the composite structure 620C. The elastic shell, which has a different compressive modulus of elasticity than the composite structure, may be formed of the same elastic material as employed in composite structure 620C and/or elastic region 610C, or a different elastic material chosen, for example, from the above-noted list of elastic materials employable as the elastic core component in the embodiment of FIG. 5. In the embodiment of FIG. 6C, the upper and lower surfaces of cylindrical-shaped core 610C, composite structure 620C and elastic shell 630C are in opposing relation to respective endplates of the first and second vertebral bodies defining intervertebral disc space 601.

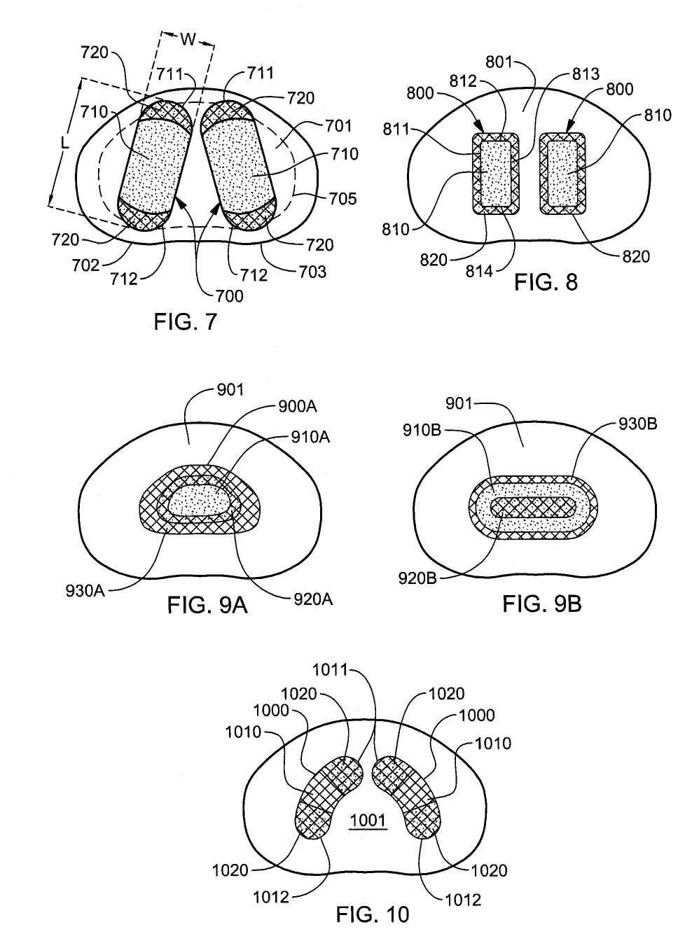

FIG. 7 illustrates an alternate embodiment wherein a bilateral approach is employed for posteriorally implanting two intervertebral prosthetic devices 700 into an intervertebral disc space 701. In this embodiment, intervertebral prosthetic devices 700 are capsule-shaped, however, as described above, other shapes such as kidney, oval, oblong, rectangular, semi-circular, semi-toroidal, trapezoidal, triangular, spherical, ellipsoidal, etc., are also contemplated (either with or without convex upper and lower surfaces). The implant process proceeds as described above in connection with FIG. 5. Specifically, an appropriate incision is made in the patient's back to form a posterior unilateral opening on each lateral side 702, 703 of the intervertebral disc space 701. The opening may be any size required to accept a single intervertebral prosthetic device configured as described herein. Through this opening, instrumentation may be inserted to evacuate remaining disc tissue. Instrumentation may also be inserted to mill or otherwise dislocate bone to fashion a path, track or recess in one or both of the vertebral endplates adjacent to the intervertebral disc space. It is understood that in certain embodiment, no bone removal may be needed. The disc space may be extracted through the milling procedure and/or subsequent insertion procedures.

The intervertebral prosthetic devices each have an elastic body component 710, and a composite structure 720 disposed at a first end 711 and a second end 712 thereof. Composite structures 720 have a higher modulus than elastic body component 710. As noted above, elastic body component 710 comprises an elastic only material similar to that described above in connection with elastic core component 510 of the embodiment of FIG. 5, while composite structure 720 comprises a composite structure, such as composite structure 520 described above in connection with the embodiment of FIG. 5.

In this embodiment, the intervertebral prosthetic devices each have a length L that extends upon cortical bone of opposing sides of an apophyseal ring 705 of a corresponding vertebral body of at least one of the first and second vertebral bodies defining the intervertebral disc space 701 within which intervertebral prosthetic device 700 is implanted. The prosthetic devices further have a width W that is smaller than this length L. By way of specific example, each prosthetic device may have a length L in a range of 18-30 mm, and a width W less than 15 mm. Additionally, the overall height of the intervertebral prosthetic device 700 may be in the range of 8-18 mm. Although not shown, the superior and inferior surfaces of the intervertebral prosthetic device may also be convex to mate with a concavity within a respective vertebral endplate of the adjacent vertebral bodies when inserted within an intervertebral disc space. As with the above embodiments, the structures of the monolithic intervertebral prosthetic device are non-articulating relative to each other. If formed separately, then the structures described herein may be attached or laminated to each other to achieve this non-articulation.

FIG. 8 illustrates another embodiment of an intervertebral prosthetic device, generally denoted 800, in accordance with an aspect of the present invention. Intervertebral prosthetic device 800 is similar to intervertebral prosthetic device 700 of FIG. 7, except that each bilaterally inserted intervertebral prosthetic device 800 within the intervertebral disc space 801 has a composite structure 820 which wraps around a rectangular-shaped elastic body component 810 and is (for example) 1-4 mm thick. Specifically, composite structure 820 wraps around elastic body component 810 to cover a first side 811, a first end 812, a second side 813 and a second end 814 thereof, with the upper surface and lower surface of the elastic body component 810 and composite structure 820 remaining uncovered. The composite structure again includes a textile structure embedded within an elastic only material, and has a higher compressive modulus of elasticity than the elastic body component 810 to enhance device support when the intervertebral prosthetic device is in operable position within the intervertebral space between adjacent vertebral bodies with the upper and lower surfaces of the elastic body component and of composite structure in opposing relation to the endplates of the vertebral bodies. The composite structure wrap-around configuration of FIG. 8 may again be employed with elastic bodies formed of different shapes, such as those noted above.

FIGS. 9A & 9B depict further, alternate embodiments of an intervertebral prosthetic device, in accordance with aspects of the present invention. In these embodiments, a lateral or anterior insertion approach may be employed.

FIG. 9A illustrates an embodiment of an intervertebral prosthetic device 900A disposed within an intervertebral disc space 901. Intervertebral prosthetic device 900A includes an elastic body or core 910A, which is an elongate structure that at least partially replicates the elongate width of the intervertebral disc space 901. Wrapped around elastic core 910A is a composite structure 920A, again comprising a textile structure embedded within an elastic material. Composite structure 920A might have a thickness in the range of 2-10 mm. A porous textile structure 930A wraps around composite structure 920A, and in this embodiment, has a thickness greater than the thickness of composite structure 920A. In addition to allowing for soft tissue in-growth, porous textile structure 930A can be enhanced with a biocompatible and osteoconductive material, or alternatively, an osteoinductive coating to facilitate bony in-growth, and thereby fixation of the intervertebral prosthetic device to, e.g., the respective upper and lower bony endplates of the vertebral bodies. As with the previous embodiment, the upper and lower surfaces of elastic body 910A, composite structure 920A and porous textile structure 930A are in opposing relation to the respective endplates of the upper and lower vertebral bodies when the intervertebral prosthetic device is disposed within the intervertebral space.

FIG. 9B illustrates an alternative embodiment of an intervertebral prosthetic device 900B, wherein the composite structure 920B is the core around which an elastic region 910B wraps, leaving exposed upper and lower surfaces of composite structure 920B as illustrated. A porous textile structure 930B wraps around elastic body 910B to complete the intervertebral prosthetic device. If desired, regions of porous textile structure 930B may be coated with a biological factor to facilitate either soft tissue in-growth or bony in-growth, and thereby fixation of the intervertebral prosthetic device to, for example, the upper and lower vertebral bodies and the disc annulus.

FIG. 10 illustrates another embodiment of an intervertebral prosthetic device 1000, in accordance with an aspect of the present invention. In this figure, two intervertebral prosthetic devices 1000 are illustrated within an intervertebral disc space 1001 defined between adjacent vertebral bodies (now shown). By way of example, intervertebral prosthetic device 1000 could have been posterially laterally inserted, for example as described above in connection with the embodiments of FIGS. 7 & 8. In the embodiment of FIG. 10, the intervertebral prosthetic device has a body component 1010 which is a porous textile structure. Formed integral with this porous textile structure are composite structures 1020 disposed at a first end 1011 and a second end 1012 of each body component. In this implementation, the prosthetic devices are kidney-shaped, which is by way of example only. Other shapes such as oval, oblong, semi-circular, semi-toroidal, trapezoidal, triangular, rectangular, spherical, ellipsoidal, etc. are also contemplated (either with or without convex upper and lower surfaces). Further, as with the above-described embodiments, the porous textile structure may be coated with a biological factor to promote bony in-growth and/or to include other therapeutic agents as described above.

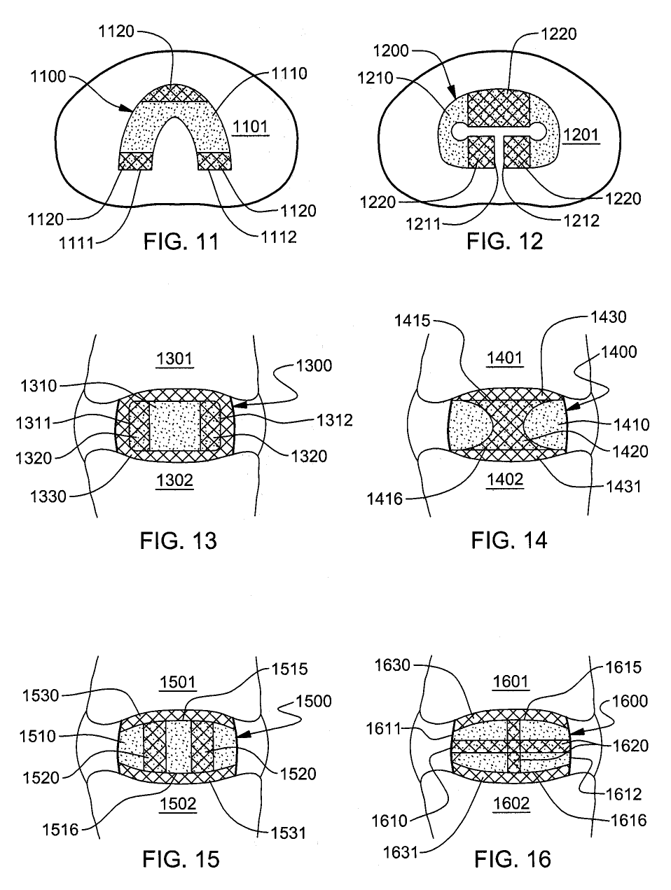

FIG. 11 depicts an intervertebral prosthetic device 1100 disposed within an intervertebral disc space 1101, wherein an elastic body component 1110 is a general U-shaped structure having a first end 1111 and a second end 1112. A composite structure 1120 is disposed at first end 1111 and at second end 1112, as well as at a portion of the bend in the U-shaped structure as illustrated. As with the above embodiments, the elastic body component comprises an elastic material similar to that described above in connection with elastic core component 510 of the embodiment of FIG. 5, while composite structure 1120 comprises a composite structure such as composite structure 520 described above in connection with the embodiment of FIG. 5. Specifically, composite structure 1120 is a textile structure embedded within an elastic material, as described. This composite structure has a higher compressive modulus of elasticity than the elastic body component and provides enhanced device support when the intervertebral prosthetic device is in operable position within an intervertebral space between adjacent vertebral bodies.

FIG. 12 depicts a further embodiment of an intervertebral prosthetic device 1200 disposed within intervertebral disc space 1201. In this embodiment, the intervertebral prosthetic device comprises a folded implant having shape memory so that it can be unfolded for implantation, yet returned to its folded configuration when relaxed in the disc space, as illustrated. The implant has two arms that are folded over to create an inner fold. The arms preferably abut one another at their ends 1211, 1212 when in the folded configuration illustrated and also abut the middle portion of the implant. This creates an implant having a substantially solid center core and provides the support necessary to avoid compression of the prosthetic device in most patients.

In the embodiment illustrated, the body of the prosthetic device is an elastic structure 1210, with multiple composite structures 1220 integrated therein. Specifically, a first composite structure 1220 exists at first end 1211, a second composite structure 1220 exists at second end 1212 and a third composite structure 1220 exists within the center core of elastic body component 1210. The elastic body component 1210 and composite structures 1220 may comprise structures and materials as described above in connection with elastic core component 510 and composite structure 520 of the embodiment of FIG. 5.

As an enhancement, the illustrated implant may have external side surfaces that include at least one grove extending along the surface thereof to advantageously relieve compressive force on the external side of the implant when the implant is deformed into a substantially straightened, or otherwise unfolded configuration for insertion into the intervertebral disc space. This allows excessive short-term deformation without permanent deformation, cracks, tears or other breakage. For example, the implant may include a plurality of grooves disposed along its external surface, with the grooves typically extending from the top surface to the bottom surface of the implant. By way of example, at least one groove may be disposed on either side of the prosthetic device.

In one method, an implant instrument such as described in the above-incorporated application entitled “Instruments And Methods For Implanting Nucleus Replacement Material In An Intervertebral Disc Nucleus Space” may be employed. When a shape memory implant is employed, the method may include the step of unfolding the implant so it assumes a “straightened” configuration in the insert instrument. The implant may then be delivered via the inserter through a hole in the disc annulus. After implantation, the implant returns naturally to its relaxed, folded configuration that mimics the shape of a natural disc. In this folded configuration, the implant is too large be expelled through the insertion hole.

FIGS. 13-16 are lateral views of further embodiments of intervertebral prosthetic devices, in accordance with aspects of the present invention.

Referring first to FIG. 13, an intervertebral prosthetic device 1300 is shown disposed between a first vertebral body 1301 and a second vertebral body 1302. In this embodiment, the core or body region is a unitary elastic structure 1310, similar to the elastic core component described above in connection with FIG. 5. Further, a composite structure 1320 is disposed at a first end 1311 and a second end 1312 of the core component. This composite structure, which includes a textile structure embedded within an elastic material, has a higher compressive modulus of elasticity than the unitary elastic structure 1310, thereby providing enhanced device support when the device is in an operable position within an intervertebral disc space between first and second vertebral bodies 1301, 1302. A porous textile structure 1330 (e.g., 1-4 mm thick) surrounds the composite structure 1320 and elastic core 1310. Together, the porous textile structure 1330, composite structure 1320 and elastic core component 1310 are sized to at least partially fill the intervertebral space when an operable position therein with a first portion of the porous textile structure engaging the first vertebral body 1301 and a second portion of the porous textile structure engaging the second vertebral body 1302. As in the embodiments described above, a biological factor or other therapeutic agent may be employed within the porous textile structure to facilitate, for example, soft tissue in-growth or bony fixation of the prosthetic device (to the adjacent vertebral bodies).

FIG. 14 depicts a further embodiment of an intervertebral prosthetic device 1400 disposed between a first vertebral body 1401 and a second vertebral body 1402. In this embodiment, an elastic core component 1410 is again provided, within which a composite structure 1420 is disposed. Composite structure 1420 is shown to extend between a first, upper surface 1415 of elastic core component 1410 and a second lower surface 1416 of elastic core component 1410 (and extends a height therebetween, e.g., in the range of 7-10 mm). Further, in the embodiment illustrated, the composite structure 1420 is tapered in a center region thereof as illustrated. A first porous textile structure 1430 and a second porous textile structure 1431 are provided at the upper and lower surfaces of the elastic core component to facilitate, for example, bony fixation of the prosthetic device to the adjacent vertebral bodies, as well as distribution of applied pressure throughout the prosthetic device.

FIG. 15 is a further alternate embodiment wherein an intervertebral prosthetic device 1500 is disposed between a first vertebral body 1501 and a second vertebral body 1502. In this embodiment, intervertebral prosthetic device 1500 includes an elastic core 1510 and multiple composite structures 1520 disposed therein extending between an upper surface 1515 and a lower surface 1516 of the elastic core component. Porous textile structures 1530, 1531 again interface the intervertebral prosthetic device to the first and second vertebral bodies 1501, 1502.

FIG. 16 illustrates a further embodiment of an intervertebral prosthetic device 1600 disposed between a first vertebral body 1601 and a second vertebral body 1602. In this implementation, one composite structure 1620 extends between an upper surface 1615 and a lower surface 1616 of the elastic core component 1610, and a second composite structure 1620 extends transverse to the first composite structure between a first end 1611 and a second end 1612 of the elastic core component.

As a further alternative, the intervertebral prosthetic device embodiments of FIGS. 5-16 could each be modified to remove the elastic region and substitute therefor the composite structure, which includes a textile structure embedded within the elastic material, either with or without the porous textile structures illustrated in the various figures. For example, FIG. 5 would have a body component comprising only the composite structure with the porous textile structures remaining as vertebral endplate contacting surfaces. In the embodiment of FIGS. 6A-6C, a cylindrical body component is provided, again comprising only the composite structure having a central longitudinal axis which intersects the vertebral endplates of the adjacent vertebral bodies when the intervertebral prosthetic device is implanted in operable position within an intervertebral space between vertebrae. The resulting structures of FIGS. 7-16 will be apparent to one skilled in the art from the above-noted discussion.

Although certain preferred embodiments have been depicted and described in detail herein, it will be apparent to those skilled in the relevant art that various modifications, additions and substitutions can be made without departing from the concepts disclosed and therefore these are to be considered to be within the scope of the following claims. For example, although the devices and methods of the present invention are particularly applicable to the lumbar region of the spine, it should nevertheless be understood that the present invention is also applicable to other portions of the spine, including the cervical or thoracic regions of the spine.

What is claimed is:

1. An intervertebral prosthetic device comprising:
a body component configured for implantation within an intervertebral space defined between a first vertebral body and a second vertebral body, the body component comprising a composite structure including a textile structure embedded within an elastic material; and
a core component disposed within the body component, the core component comprising one of a spherical-shaped elastic structure or a cylindrical-shaped elastic structure, wherein the body component has a higher compressive modulus of elasticity than the core component to enhance device support when the intervertebral prosthetic device is in operable position within an intervertebral space between the first and second vertebral bodies.

2. The intervertebral prosthetic device of claim 1, wherein the core component comprises a same elastic material as employed in the composite structure.

3. The intervertebral prosthetic device of claim 1, further comprising a porous textile structure at least partially covering the body component, the porous textile structure, body component and core component being configured to facilitate implantation of the intervertebral prosthetic device within the intervertebral space between the first and second vertebral bodies in an operable position with at least a portion of the porous textile structure contacting at least one endplate of the first vertebral body and the second vertebral body.

4. The intervertebral prosthetic device of claim 3, wherein the at least a portion of the porous textile structure contacting at least one endplate of the first vertebral body and second vertebral body is coated with a biological factor to facilitate bony fixation of the intervertebral prosthetic device to the at least one endplate of the first vertebral body and second vertebral body.

5. The intervertebral prosthetic device of claim 1, wherein the core component is the cylindrical-shaped elastic structure, the body component is a ring-shaped structure surrounding the core component and wherein the intervertebral prosthetic device further comprises a ring-shaped elastic structure surrounding at least a portion of the body component, the ring-shaped elastic structure having a different compressive modulus of elasticity than the body component.

6. The intervertebral prosthetic device of claim 1, wherein the textile structure within the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

7. The intervertebral prosthetic device of claim 6, wherein density of the textile structure within the composite structure at least partially varies across the composite structure.

8. The intervertebral prosthetic device of claim 6, wherein volume of the elastic material within the composite structure at least partially varies across the composite structure.

9. The intervertebral prosthetic device of claim 1, further comprising a porous textile structure at least partially covering the body component, the porous textile structure being disposed to contact at least one endplate of the first vertebral body and the second vertebral body when the intervertebral prosthetic device is in operable position within the intervertebral space, and wherein a pharmacological agent is disposed within the portion of the porous textile structure to contact the at least one endplate of the first vertebral body and the second vertebral body, the pharmacological agent comprising at least one of a growth factor or an agent effective for treating at least one of a degenerative disc disease, spinal arthritis, spinal infection, spinal tumor or osteoporosis.

10. An intervertebral prosthetic device comprising:
a body component configured for implantation within an intervertebral space defined between a first vertebral body and a second vertebral body;
a core component disposed within the body component, the core component comprising a cylindrical-shaped structure having a longitudinal axis extending in a direction which intersects endplates of the first vertebral body and second vertebral body when the intervertebral prosthetic device is disposed in operable position within the intervertebral space; and
wherein one of the core component and the body component is an elastic structure, and the other of the core component and body component is a composite structure, the composite structure comprising a textile structure embedded within an elastic material, and wherein the composite structure has a higher compressive modulus of elasticity than the elastic structure to enhance device support when in an operable position within the intervertebral space between the first vertebral body and the second vertebral body.

11. The intervertebral prosthetic device of claim 10, wherein the core component is the elastic structure, the body component is the composite structure, and wherein the intervertebral prosthetic device further comprises an elastic layer at least partially surrounding the composite structure, the elastic layer having a different modulus than the composite structure.

12. The intervertebral prosthetic device of claim 10, wherein the elastic structure, the elastic layer, and elastic material of the composite structure comprise a same elastic material.

13. The intervertebral prosthetic device of claim 10, wherein the core component is the composite structure, the body component is the elastic structure, and wherein end surfaces of the core component are disposed in opposing relation to an endplate of the respective first and second vertebral bodies when the intervertebral prosthetic device is in operable position within the intervertebral space.

14. The intervertebral prosthetic device of claim 10, wherein the textile structure of the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

15. The intervertebral prosthetic device of claim 14, wherein density of the textile structure within the composite structure at least partially varies across the composite structure.

16. The intervertebral prosthetic device of claim 10, wherein modulus of at least one of the elastic structure or the composite structure at least partially varies within the intervertebral prosthetic device.

17. The intervertebral prosthetic device of claim 10, wherein the elastic structure comprises an elastomeric material selected from the group consisting of silicones, polyurethanes, copolymers of silicone and polyurethane, polyolefins, hydrogels, polyisobutylene rubber, polyisoprene rubber, neoprene rubber, nitrile rubber, polyolefin rubber and vulcanized rubber.

18. An intervertebral prosthetic device comprising:
a body component comprising an elastic structure having a first side, a first end, a second side, a second end, an upper surface and a lower surface, the upper surface and the lower surface being disposed in opposing relation to a respective endplate of the first vertebral body and the second vertebral body defining an intervertebral space when the intervertebral prosthetic device is disposed in operable position within the intervertebral space; and
a composite structure wrapping around the body component to cover the first side, first end, second side and second end thereof, wherein the upper surface and lower surface of the body component are uncovered by the composite structure, the composite structure comprising a textile structure embedded within an elastic material, and wherein the composite structure has a higher compressive modulus of elasticity than the elastic structure to enhance device support when the intervertebral prosthetic device is in operable position within the intervertebral space between the first and second vertebral bodies with the upper and lower surfaces of the body component in opposing relation to the endplates of the first and second vertebral bodies.

19. The intervertebral prosthetic device of claim 18, wherein the elastic structure is an elongate elastic structure.

20. The intervertebral prosthetic device of claim 19, wherein elasticity of the elongate elastic structure at least partially progressively varies between the first and the second end thereof.

21. The intervertebral prosthetic device of claim 18, wherein the textile structure within the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

22. The intervertebral prosthetic device of claim 18, further comprising a porous textile structure at least partially covering the composite structure, the porous textile structure having a different compressive modulus of elasticity than the composite structure, wherein the porous textile structure, composite structure and body component are configured to facilitate implantation of the intervertebral prosthetic device within the intervertebral space between the first and second vertebral bodies in an operable position with at least a portion of the porous textile structure contacting at least one endplate of the first vertebral body and the second vertebral body defining the intervertebral space.

23. The intervertebral prosthetic device of claim 22, wherein the porous textile structure wraps around the composite structure and has a thickness greater than a thickness of the composite structure.

24. The intervertebral prosthetic device of claim 22, wherein the textile structure within the composite structure, and the porous textile structure each comprise at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole, and wherein the elastic structure comprises an elastomeric material selected from the group consisting of silicones, polyurethanes, copolymers of silicone and polyurethane, polyolefins, hydrogels, polyisobutylene rubber, polyisoprene rubber, neoprene rubber, nitrile rubber, polyolefin rubber and vulcanized rubber.

25. The intervertebral prosthetic device of claim 24, wherein the porous textile structure further comprises a pharmacological agent comprising at least one of a growth factor or an agent effective for treating a generative disc disease, spinal arthritis, spinal infection, spinal tumor or osteoporosis.

26. An intervertebral prosthetic device comprising:
a body component having at least a first end and a second end, the body component comprising an elastic structure; and
multiple composite structures, one composite structure of the multiple composite structures being disposed at the first end of the body component, and one composite structure of the multiple composite structures being disposed at the second end of the elastic body component, each composite structure of the multiple composite structures comprising a textile structure embedded within an elastic material, and wherein the multiple composite structures have a higher compressive modulus of elasticity than the body component to enhance device support when in an operable position within an intervertebral space between a first vertebral body and a second vertebral body.

27. The intervertebral prosthetic device of claim 26, wherein the body component is an elongate elastic structure, and wherein the first end and the second end thereof are opposing ends intersecting a longitudinal axis of the body component.

28. The intervertebral prosthetic device of claim 26, wherein the body component is an elongate elastic structure having a length that extends upon cortical bone of opposing sides of an apophyseal ring of at least one of the first and second vertebral bodies, and a width that is smaller than the length.

29. The intervertebral prosthetic device of claim 26, wherein the body component is a U-shaped elastic structure, and wherein the multiple composite structures further comprise one composite structure of the multiple composite structures disposed within at least a portion of a bend in the U-shaped elastic structure.

30. The intervertebral prosthetic device of claim 26, further comprising a porous textile structure surrounding the body component and the multiple composite structures, the porous textile structure having a different modulus than the composite structure, wherein the porous textile structure, multiple composite structures and body component are configured to facilitate implantation of the intervertebral prosthetic device within the intervertebral space between the first and second vertebral bodies in an operable position with at least a portion of the porous textile structure contacting at least one endplate of the first vertebral body and second vertebral body.

31. The intervertebral prosthetic device of claim 30, wherein the at least a portion of the porous textile structure contacting the at least one endplate of the first vertebral body and second vertebral body comprises at least one of a growth factor or an agent effective for treating a degenerative disc disease, spinal arthritis, spinal infection, spinal tumor or osteoporosis.

32. An intervertebral prosthetic device comprising:
an elastic core component;
a composite structure at least partially surrounding the elastic core component, the composite structure comprising a textile structure embedded within an elastic material, wherein the composite structure has a higher compressive modulus of elasticity than the elastic core component to enhance device support when in operable position within an intervertebral space between a first vertebral body and a second vertebral body; and
a porous textile structure at least partially covering the composite structure, the porous textile structure having a different modulus than the composite structure, wherein the porous textile structure, composite structure and elastic core component are configured to facilitate implantation of the intervertebral prosthetic device within the intervertebral space between the first and second vertebral bodies in an operable position with at least a portion of the porous textile structure contacting at least one endplate of the first vertebral body and second vertebral body.

33. The intervertebral prosthetic device of claim 32, wherein the elastic core component comprises a same elastic material as the elastic material employed in the composite structure.

34. The intervertebral prosthetic device of claim 32, wherein the textile structure of the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

35. The intervertebral prosthetic device of claim 32, wherein the porous textile structure surrounds the composite structure and elastic core component, with a first portion thereof contacting a first endplate of the first vertebral body and a second portion thereof contacting a second endplate of the second vertebral body when the intervertebral prosthetic device is disposed within the intervertebral space between the first and second vertebral bodies.

36. The intervertebral prosthetic device of claim 32, wherein at least a portion of the porous textile structure comprises at least one of a growth factor or an agent effective for treating a generative disc disease, spinal arthritis, spinal infection, spinal tumor or osteoporosis.

37. The intervertebral prosthetic device of claim 32, wherein the textile structure within the composite structure comprises a same textile structure as employed within the porous textile structure.

38. The intervertebral prosthetic device of claim 32, wherein density of the textile structure within the composite structure is different than density of the porous textile structure.

39. An intervertebral prosthetic device comprising:
a porous textile structure having at least a first end and a second end; and
multiple composite structures, one composite structure of the multiple composite structures being disposed at the first end of the porous textile structure, and another composite structure of the multiple composite structures being disposed at the second end of the porous textile structure, each composite structure of the multiple composite structures comprising a textile structure embedded within an elastic material, wherein the multiple composite structures have a different compressive modulus of elasticity than the porous textile structure to enhance device support when in an operable position within an intervertebral space between a first vertebral body and a second vertebral body.

40. The intervertebral prosthetic device of claim 39, wherein the porous textile structure is an elongate structure, and wherein the textile structures within the multiple composite structures comprise a same textile structure as employed within the porous textile structure.

41. The intervertebral prosthetic device of claim 39, wherein a portion of the porous textile structure contacts at least one endplate of the first vertebral body and the second vertebral body when the intervertebral prosthetic device is in operable position within the intervertebral space.

42. The intervertebral prosthetic device of claim 39, wherein the textile structure within the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

43. The intervertebral prosthetic device of claim 39, wherein the porous textile structure comprises at least one of a growth factor or an agent effective for treating a degenerative disc disease, spinal arthritis, spinal infection, spinal tumor or osteoporosis.

44. The intervertebral prosthetic device of claim 39, wherein density of the porous textile structure is different than density of the textile structures within the multiple composite structures.

45. An intervertebral prosthetic device comprising:
an elastic body component configured for implantation within an intervertebral space defined between a first vertebral body and a second vertebral body; and
at least one composite structure extending through the elastic body component between a first surface and a second surface thereof, each composite structure comprising a textile structure embedded within an elastic material, wherein the at least one composite structure has a higher compressive modulus of elasticity than the elastic body component to enhance intervertebral prosthetic device support when in an operable position within the intervertebral space between the first and second vertebral bodies.

46. The intervertebral prosthetic device of claim 45, wherein the at least one composite structure comprises at least one centrally-disposed composite structure extending between an upper surface and a lower surface of the elastic body component.

47. The intervertebral prosthetic device of claim 46, wherein the at least one composite structure is tapered in a center region thereof.

48. The intervertebral prosthetic device of claim 45, further comprising multiple composite structures disposed within the elastic body component, wherein at least one composite structure of the multiple composite structures extends between an upper surface and a lower surface of the elastic body component.

49. The intervertebral prosthetic device of claim 48, wherein at least one composite structure of the multiple composite structures extends between a first end and a second end of the elastic body component in a direction substantially transverse to the at least one composite structure extending between the upper surface and lower surface of the elastic body component.

50. The intervertebral prosthetic device of claim 49, further comprising a first porous textile structure disposed at the upper surface of the elastic body component and a second porous textile structure disposed at the lower surface of the elastic body component, the first and second porous textile structures having a different modulus than the at least one composite structure, and wherein the first and second porous textile structures are coated with a biological factor to facilitate bony fixation of the intervertebral prosthetic device to the first and second vertebral bodies when the intervertebral prosthetic device is implanted in operable position within the intervertebral space.

51. The intervertebral prosthetic device of claim 45, further comprising a first porous textile structure disposed at an upper surface of the elastic body component and a second porous textile structure disposed at a lower surface of the elastic body component, the first and second porous textile structures being fabricated of a biocompatible material, and having a different modulus than the at least one composite structure, and wherein the intervertebral prosthetic device is sized to at least partially fill the intervertebral space when in operable position therein with the first and second porous textile structures engaging the first vertebral body and the second vertebral body, respectively.

52. The intervertebral prosthetic device of claim 45, further comprising a porous textile structure surrounding the elastic body component, the porous textile structure being fabricated of a biocompatible material, and wherein the intervertebral prosthetic device is sized to at least partially fill the intervertebral space when in operable position therein with the porous textile structure engaging the first vertebral body and the second vertebral body.

53. An intervertebral prosthetic device comprising:
a load-bearing elastic body component having shape memory, the elastic body component being in a first, folded configuration effective to serve as a prosthetic disc nucleus, wherein the elastic body component is configurable into a second, straightened configuration for insertion through an opening in an intervertebral disc annulus fibrous, wherein the shape memory is effective to return the elastic body component to its first, folded configuration after the elastic body component is straightened to its second, straightened configuration and inserted into an intervertebral space; and
multiple composite structures, one composite structure being disposed at a first end of the elastic body component, and one composite structure being disposed at a second end of the elastic body component, each composite structure comprising a textile structure embedded within an elastic material, and wherein the composite structures have a higher modulus than the elastic body component to enhance prosthetic device support when in an operable position within an intervertebral space between a first vertebral body and a second vertebral body.

54. The intervertebral prosthetic device of claim 53, wherein the multiple composite structures further comprise a composite structure disposed at a center region of the elastic body component intermediate the first end and the second end thereof.

55. The intervertebral prosthetic device of claim 53, wherein the textile structure within the composite structure comprises at least one of a woven, knitted, braided, or non-woven structure employing at least one of fabric, polymeric, ceramic or metallic filaments, the polymeric filaments comprising one or more of polyester, polyethyleneterephthalate (PET), polyethylene, ultra-high molecular-weight polyethylene (UHMWPE), polyaryletherketone, polyetheretherketone (PEEK), polypropylene, polyamide, acetate, acrylic, aramid, elastoester or polybenzimidazole.

56. The intervertebral prosthetic device of claim 55, wherein the elastic body component and the elastic material within the multiple composite structures comprise a same elastomeric material, the elastomeric material being selected from the group consisting of silicones, polyurethanes, copolymers of silicones and polyurethane, polyolefins, hydrogels, polyisobutylene rubber, polyisoprene rubber, neoprene rubber, nitrile rubber, polyolefin rubber and vulcanized rubber.

57. An intervertebral prosthetic device comprising:
a body component configured for implantation within an intervertebral space defined between a first vertebral body and a second vertebral body; and
wherein the body component comprises a composite structure including a textile structure embedded within an elastic material, wherein the textile structure within the composite structure comprises at least one of a woven, knitted, braided, or non-woven structure employing at least one of fabric, polymeric, ceramic or metallic filaments.

58. The intervertebral prosthetic device of claim 57, further comprising a porous textile structure at least partially covering the body component, the porous textile structure and body component being configured to facilitate implantation of the intervertebral prosthetic device within the intervertebral space between the first and second vertebral bodies in an operable position with at least a portion of the porous textile structure contacting at least one endplate of the first vertebral body and the second vertebral body.

59. The intervertebral prosthetic device of claim 58, wherein the at least a portion of the porous textile structure contacting at least one endplate of the first vertebral body and the second vertebral body is coated with a biological factor to facilitate bony fixation of the intervertebral prosthetic device to the at least one endplate of the first vertebral body and the second vertebral body.
